SEQUENCE LISTING (14 sequences)

SEQ ID NO: 1 - DNA, 7599 bp, synthetic construct
  source 1..7599: mol_type = other DNA
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccttaaccctagaaagatagtctgcgtaaaattgacgcatgcattcttgaaatattgctctctctttctaaatagcgcgaatccgtcgctgtgcatttaggacatctcagtcgccgcttggagctcccgtgaggcgtgcttgtcaatgcggtaagtgtcactgattttgaactataacgaccgcgtgagtcaaaatgacgcatgattatcttttacgtgacttttaagatttaactcatacgataattatattgttatttcatgttctacttacgtgataacttattatatatatattttcttgttatagatatcgtcgacgcggccgctaagatacattgatgagtttggacaaaccacaactagaatgcagtgaaaaaaatgctttatttgtgaaatttgtgatgctattgctttatttgtaaccattataagctgcaataaacaagttatttaaattcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgaacggccctggggacgtcgtcgcgggtggcgaggcgcaccgtgggcttgtactcggtcatatcgatggggccgggattttcctccacgtccccgcatgttagaagacttcccctgccctctccgcttccgttaaccccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacatggggggatgtacagtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgcataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcctgcgcccttcgtctgacgtggcagcgctcgccgtgaggaggggggcgcccgcgggaggcgccaaaacccggcgcggaggccactagtgaattcgagcggaggccttacgtaattaccctgttatccctaccgcgggtaacggagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaaggtctagagctagcgccaccatgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctagctcgagggcgcgcccgctgatcagcctcgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggggatcctttgttactttatagaagaaattttgagtttttgtttttttttaataaataaataaacataaataaattgtttgttgaatttattattagtatgtaagtgtaaatataataaaacttaatatctattcaaattaataaataaacctcgatatacagaccgataaaacacatgcgtcaattttacgcatgattatctttaacgtacgtcacaatatgattatctttctagggttaaagagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 2 - DNA, 819 bp, Macaca fascicularis
  source 1..819: mol_type = other DNA
atgttcgtcaaatccgagaccttggagttgaaggaggaagaggacgtgttggtgctgctcggttcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgcggctgcgggtgcggagggctgccggcccgcacggctgctgggtctggtgcacgattgcaaacggcgcccctctcgggcgcgggccatctcccgaggcgccaagaccgcggagacggtgcagcgcatcaagaagacccgtagactgaaggccaacaaccgcgagcgaaaccgcatgcacaacctcaacgcggcactggacgcgctgcgcgaggtgctccccacgttcccagaggacgccaagctcaccaagatcgagaccctgcgcttcgcccacaactacatctgggcgctcaccgagaccctgcgcctggcggatcactgcgggggcggcggcggaggcctgccgggggcgctcttctccgaggcagtgttgctgagcccgggaggcgccagcgccaccctgagcagcagcggagacagcccctcgcccgcctccacgtggagttgcaccaacagccccgcgccgtcctcctccgcgtcctccaactccacctccccctacagctgcactttatcgcccgccagcccggccgggtcagacatggactattggcagcccccacctcccgacaagcaccgctatgcacctcacctccccatagccagggattgtatctag

SEQ ID NO: 3 - DNA, 826 bp, Homo sapiens
  source 1..826: mol_type = other DNA
atgttcgtcaaatccgagaccttggagttgaaggaggaagaggacgtgttagtgctgctcggatcggcctcccccgccttggcggccctgaccccgctgtcatccagcgccgacgaagaagaggaggaggagccgggcgcgtcaggcggggcgcgtcggcagcgcggggctgaggccgggcagggggcgcggggcggcgtggctgcgggtgcggagggctgccggcccgcacggctgctgggtctggtacacgattgcaaacggcgcccttcccgggcgcgggccgtctcccgaggcgccaagacggccgagacggtgcagcgcatcaagaagacccgtagactgaaggccaacaaccgcgagcgaaaccgcatgcacaacctcaacgcggcactggacgcgctgcgcgaggtgctccccacgttccccgaggacgccaagctcaccaagatcgagaccctgcgcttcgcccacaactacatctgggcactcaccgagaccctgcgcctggcggatcactgcgggggcggcggcgggggcctgccgggggcgctcttctccgaggcagtgttgctgagcccgggaggagccagcgccgccctgagcagcagcggagacagcccctcgcccgcctccacgtggagttgcaccaacagccccgcgccgtcctcctccgtgtcctccaattccacctccccctacagctgcactttatcgcccgccagcccggccgggtcagacatggactattggcagcccccacctcccgacaagcaccgctatgcacctcacctccccatagccagggattgtatctaggctcgag

SEQ ID NO: 4 - DNA, 681 bp, synthetic construct
  source 1..681: mol_type = other DNA
atgcccgccatgaagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagacccccatcgccttcgccagatctcgagtctag

SEQ ID NO: 5 - DNA, 379 bp, synthetic construct
  source 1..379: mol_type = other DNA
gagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaa

SEQ ID NO: 6 - DNA, 1212 bp, synthetic construct
  source 1..1212: mol_type = other DNA
gtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcaccgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggcc

SEQ ID NO: 7 - DNA, 600 bp, synthetic construct
  source 1..600: mol_type = other DNA
tcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgagacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgaacggccctggggacgtcgtcgcgggtggcgaggcgcaccgtgggcttgtactcggtcat

SEQ ID NO: 8 - DNA, 589 bp, synthetic construct
  source 1..589: mol_type = other DNA
tttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaa

SEQ ID NO: 9 - DNA, 861 bp, synthetic construct
  source 1..861: mol_type = other DNA
ttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcat

SEQ ID NO: 10 - DNA, 456 bp, synthetic construct
  source 1..456: mol_type = other DNA
aaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgt

SEQ ID NO: 11 - DNA, 105 bp, synthetic construct
  source 1..105: mol_type = other DNA
actcttcctttttcaatattattgaagcatttatcagggttattgtctcatgagcggatacatatttgaatgtatttagaaaaataaacaaataggggttccgcg

SEQ ID NO: 12 - DNA, 19 bp, synthetic construct
  source 1..19: mol_type = other DNA
taatacgactcactatagg

SEQ ID NO: 13 - DNA, 19 bp, synthetic construct
  source 1..19: mol_type = other DNA
cctttagtgagggttaatt

SEQ ID NO: 14 - DNA, 744 bp, synthetic construct
  source 1..744: mol_type = other DNA
cccggggagcatgtcaaggtcaaaatcgtcaagagcgtcagcaggcagcatatcaaggtcaaagtcgtcaagggcatcggctgggagcatgtctaagtcaaaatcgtcaagggcgtcggtcggcccgccgctttcgcactttagctgtttctccaggccacatatgattagttccaggccgaaaaggaaggcaggttcggctccctgccggtcgaacagctcaattgcttgtttcagaagtgggggcatagaatcggtggtaggtgtctctctttcctcttttgctacttgatgctcctgttcctccaatacgcagcccagtgtaaagtggcccacggcggacagagcgtacagtgcgttctccagggagaagccttgctgacacaggaacgcgagctgattttccagggtttcgtactgtttctctgttgggcgggtgccgagatgcactttagccccgtcgcgatgtgagaggagagcacagcggtatgacttggcgttgttccgcagaaagtcttgccatgactcgccttccagggggcaggagtgggtatgatgcctgtccagcatctcgattggcagggcatcgagcagggcccgcttgttcttcacgtgccagtacagggtaggctgctcaactcccagcttttgagcgagtttccttgtcgtcaggccttcgataccgactccattgagtaattccagagcagagtttatgactttgctcttgtccagtctagacat

(19) United States
(12) Patent Application Publication
(10) Pub. No.: US 2026/0337632 A1
(43) Pub. Date: Oct. 29, 2026

(54) COMPOSITIONS AND METHODS FOR INDUCED STEM CELL DIFFERENTIATION TO NEURONAL LINEAGE

(71) Applicant: Exir, Inc., Sunnyvale, CA (US)

(72) Inventor: Morteza Roodgar, Menlo Park, CA (US)

(21) Appl. No.: 19/677,360

(22) Filed: May 14, 2026

Related U.S. Application Data
(63) Continuation of application No. PCT/US24/56316, filed on Nov. 17, 2024.
(60) Provisional application No. 63/600,402, filed on Nov. 17, 2023.

(51) Int. Cl. C12N 15/85; C07K 14/47

(52) U.S. Cl. CPC C12N 15/85; C07K 14/4702

(57) ABSTRACT

A method of differentiating nonhuman primate iPSCs to neurons may include providing a modified vector comprising a piggyBac vector backbone expressing a differentiation factor gene; treating the nonhuman primate iPSCs with the modified vector in medium; replacing the medium with a second medium including BDNF, NT-3, and laminin; adding medium supplemented with L-glutamine or an L-glutamine alternative, BDNF, and NT3; and producing neuronal lineage cells.

### CROSS-REFERENCE TO RELATED APPLICATIONS

This application is a continuation of International Patent Application Ser. No. PCT/US24/56316, filed Nov. 17, 2024, which claims the priority benefit of U.S. Provisional Patent Application Ser. No. 63/600,402, filed Nov. 17, 2023, the contents of each of which are herein incorporated by reference in their entirety.

### SEQUENCE LISTING

The present application is being filed along with a Sequence Listing in electronic format. The Sequence Listing is provided as a file entitled 0138-701600 SequenceListing.xml, created Nov. 15, 2024, which is 28,000 bytes in size. The information in the electronic format of the Sequence Listing is incorporated by reference in its entirety.

### INCORPORATION BY REFERENCE

All publications and patent applications mentioned in this specification are herein incorporated by reference in their entirety, as if each individual publication or patent application were specifically and individually indicated to be incorporated by reference in its entirety.

### TECHNICAL FIELD

This disclosure relates generally to the field of cellular differentiation, and more specifically to the field of lentiviral transduction for cellular differentiation. Described herein are compositions and methods for induced stem cell differentiation.

### BACKGROUND

Induced pluripotent stem cells (iPSCs) have the potential to be differentiated into different cell types, including neuronal cells. This process has been successfully conducted using human iPSCs.

### SUMMARY

In some aspects, the techniques described herein relate to a vector for differentiating nonhuman primate iPSCs from Macaca fascicularis, wherein the vector includes a nucleic acid sequence including a sequence encoding: a piggyBac vector backbone; and a differentiation factor gene.

In some aspects, the techniques described herein relate to a vector for differentiating nonhuman primate iPSCs, wherein the vector includes a nucleic acid sequence including a sequence encoding: a piggyBac vector backbone; and a differentiation factor gene.

In some aspects, the techniques described herein relate to a vector for differentiating nonhuman primate iPSCs to neuronal lineage cells, wherein the vector includes a nucleic acid sequence including a sequence encoding: a piggyBac vector backbone; and SEQ ID NO. 2 positioned between about base 4208 and about base 5033.

In some aspects, the techniques described herein relate to a method of differentiating nonhuman primate iPSCs to neuronal lineage cells, including: providing a vector configured to express a differentiation factor gene; treating the nonhuman primate iPSCs with the vector in medium; replacing the medium with a second medium including brain-derived neurotrophic factor (BDNF), neurotrophin-3 (NT-3), and laminin; adding medium supplemented with L-glutamine, BDNF, and NT-3; and producing the neuronal lineage cells.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 shows a schematic of a piggyBac vector backbone.

FIG. 2 shows a schematic of a piggyBac vector backbone modified to express differentiation factor gene Homo sapiens NGN2 (SEQ ID NO. 3).

FIG. 3 shows a schematic of a piggyBac vector backbone modified to express differentiation factor gene Macaca fascicularis NEUROG2 (SEQ ID NO. 2).

FIG. 4 is a flow chart of an embodiment of a method for transfecting nonhuman primate (NHP) induced pluripotent stem cells (iPSCs).

FIG. 5 is a flow chart of an embodiment of a method for differentiating NHP iPSCs to an ectoderm lineage.

FIG. 6 is a flow chart showing an embodiment of a method for reprogramming nonhuman primate peripheral blood mononuclear cells (PBMCs) into iPSCs.

FIG. 7 is a schematic of an embodiment of a method for reprogramming nonhuman primate PBMCs into iPSCs.

FIG. 8 shows a fluorescence microscopy image of iPSCs from Macaca fascicularis transfected with a piggyBac vector expressing Macaca fascicularis NEUROG2 (SEQ ID NO. 2).

FIG. 9 shows quantitative polymerase chain reaction (qPCR) data of NEUROG2 expression in various cell types from, or derived from, Macaca fascicularis.

FIG. 10A shows a brightfield image of iPSC-derived iN cells at 10× magnification.

FIG. 10B shows a fluorescence microscopy image at 20× magnification of iN cells stained with 4′,6-diamidino-2-phenylindole (DAPI) and an antibody directed to neuron-specific class III beta-tubulin (TUJ1).

FIG. 10C shows a fluorescence microscopy image at 20× magnification of iN cells stained with DAPI and an antibody directed to microtubule-associated protein-2 (MAP-2).

FIG. 11 is a graph of transfection efficiency for lentiviral versus piggyBac-based transfection methods.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, is necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the claimed subject matter. Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

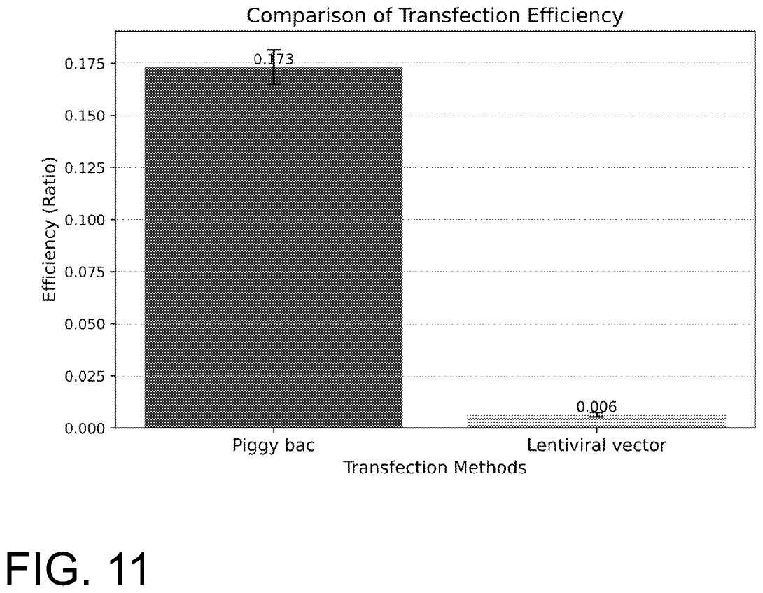

Conventionally, stem cells from humans have been differentiated using lentivirus-based systems. However, lentivirus-based systems are not efficient when differentiating NHP stem cells, for example, as shown in FIG. 11. Species-specific differences may prevent the use of human iPSC methods for nonhuman primate iPSCs. Using lentivirus-based systems, NHPs have a low transduction efficiency, for example, about 1% to about 5%. Further, using lentiviruses is undesirable since the genetic material is incorporated into the genome, which could result in mutations that may be imperceptible to the user/researcher but nonetheless impactful on the results from the experiments. Others have attempted to use Sendai virus, but these have similar drawbacks as lentiviruses. For example, Sendai virus in nonhuman primate cells is not as efficient as its usage in human cells.

The above technical problems presented by using lentivirus-based systems and/or Sendai viruses may be solved by technical solutions. For example, the systems and methods described herein may solve the above technical problems with technical solutions that include using piggyBac vectors for transfecting NHP iPSCs. Using piggyBac vectors provides several technical advantages when transfecting NHP iPSCs. For example, piggyBac vectors can be removed from the genome without leaving a footprint, and piggyBac vectors have high transposition activity in at least particular cell types. As shown and described elsewhere herein, a piggyBac vector has been modified herein to express particular differentiation factor genes to enable differentiation of NHP iPSCs into pre-defined ectoderm lineages. For example, one or more differentiation factors may be used to differentiate NHP iPSCs into NGN2 cells (iN cells). The piggyBac vectors described herein may yield a substantially higher transfection efficiency than lentivirus-based vectors. For example, the piggyBac vectors described herein may have a transfection efficiency of about 10% to about 30%, about 15% to about 20%, about 17% to about 23%, about 16% to about 20%, about 10% to about 20%, about 10% to about 25%, about 16% to about 24%, etc. The piggyBac vectors described herein may have an about ten-fold improvement or an at least about ten-fold improvement in transfection efficiency over lentivirus-based transfection systems.

As used herein, “nonhuman primates” may be used to refer to primate species that are used in genetic research or, more generally, primates that are not humans. Exemplary species, to which the methods and systems described herein may apply, include, but are not limited to, rhesus macaques (Macaca mulatta), cynomolgus macaques (Macaca fascicularis), baboons (Papio spp.), common marmosets (Callithrix jacchus), squirrel monkeys (Saimiri spp.), African green monkeys (Chlorocebus sabaeus), pigtailed macaques (Macaca nemestrina), and owl monkeys (Aotus lemurinus). In some embodiments, the systems and methods described herein may be applied to cells from cynomolgus macaques or Macaca fascicularis.

NHP iPSCs from NHP PBMCs

NHP iPSCs may be derived from NHP PBMCs. For example, a method of deriving iPSCs from NHP PBMCs may include obtaining tissue or blood from a first nonhuman primate; isolating PBMCs from the first nonhuman primate; and deriving iPSCs from the PBMCs from the first nonhuman primate.

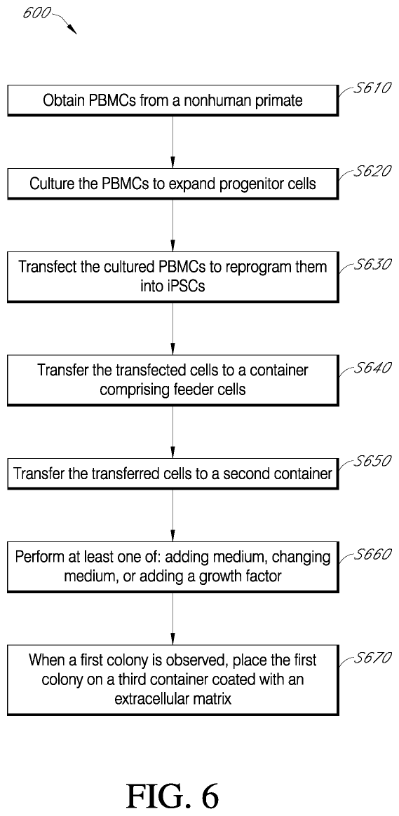

FIG. 6 shows an embodiment of deriving iPSCs from nonhuman primate PBMCs. The method 600 includes: obtaining peripheral blood mononuclear cells (PBMCs) from at least one individual of a population comprising a single species of nonhuman primate S610; culturing the PBMCs to expand blood progenitor cells (e.g., including CD34+ cells) in a hematopoietic stem cell (HSC) expansion medium for a pre-determined time period S620; transfecting the cultured PBMCs to reprogram the cultured cells into iPSCs S630; transferring the transfected cells into a container comprising a plurality of feeder cells S640; transferring the cells to a second container S650; on selected days after transfer to the second container, performing at least one of: adding medium to the cells; changing the medium in the cells; or adding one or more cell growth factors to the cells S660; and when a first cell colony appears; passaging the cells by placing each colony in a third container coated with an extracellular matrix S870. As used herein, medium may include, but is not limited to, DMEM medium, RPMI™ medium, F12™ medium, and the like. In some embodiments, specialty media are detailed below or elsewhere herein.

The method 600 may further include expanding the first colony for use in one or more of: an in vitro test, an in vivo test, or creation of a genome library for the individual primate.

In some embodiments, transfecting includes transfecting with a combination of transcription factors so that the cells are induced to overexpress the transcription factors. For example, the transcription factors may include, but not be limited to, c-myc, Klf4, Sox2, or Oct3/4. Additional or alternative transcription factors may be used and are within the scope of the present disclosure. For example, transcription factors such as Klf2, Nanog, Tfcp2l1 and Stat3 may additionally or alternatively be used.

In some embodiments, the expanding may include washing the iPSCs with a buffer (e.g., phosphate-buffered saline) and incubating the iPSCs with a cell detachment solution, for example trypsin, a chelating agent, collagenase, or the like. In some embodiments, the incubating occurs at about 37 degrees Celsius for a predetermined time period. The predetermined time period may be about 30 seconds to about 10 minutes; about 1 minute to about 60 minutes; about 1 minute to about 5 minutes; etc.

In some embodiment of method 600, transferring the transfected cells to a container with feeder cells occurs no more than about one day post-transfection. The feeder cells may comprise mouse embryonic fibroblast (MEF) cells, SNL feeder cells, or the like.

In some embodiments, transferring the cells to the second container occurs no more than about one day after transferring to the container with the feeder cells.

In some embodiments, the expanding comprises aspirating and dissociating the iPSCs into a single cell suspension.

As described elsewhere herein, the iPSCs may be differentiated into main lineages. The main lineages may include at least one of: ectoderm, mesoderm, and endoderm. Additionally, or optionally, the main lineages, for example the ectoderm lineage, may be further differentiated into final tissues, for example NGN2 neurons, astroglia, astrocytes, oligodendrocytes, and the like. The final tissues may be used for in vitro testing, autologously transplanted into the first nonhuman primate for in vivo testing, and/or used in genome library generation.

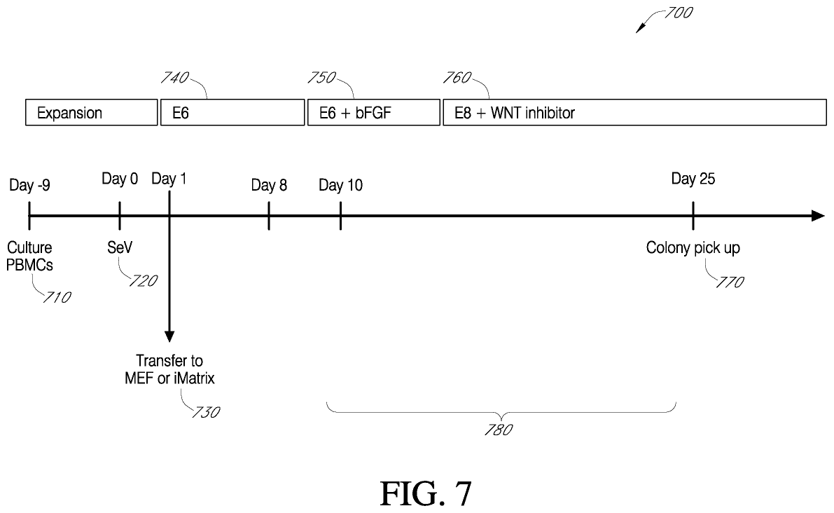

FIG. 7 shows an embodiment of a method 700 for deriving iPSCs from nonhuman primate PBMCs. For example, peripheral blood mononuclear cells (PBMCs) are obtained from an individual from a species of nonhuman primate, and the PBMCs are isolated from the animal's whole blood. The PBMCs may be isolated from the animal's whole blood using density gradient centrifugation (DGC), fluorescence-activated cell sorting, magnetic bead-based separation, buoyancy-activated cell sorting (BACS), and the like. In an embodiment, PBMCs are isolated using DGC. In an embodiment, PBMCs are isolated using BACS.

In some embodiments, a method 700 for reprogramming NHP PBMCs into iPSCs includes culturing 710 the PBMCs to expand blood progenitor cells (e.g., CD34+ cells) in a hematopoietic stem cell (HSC) expansion medium such as StemSpan™ (Stemcell Technologies Inc., Vancouver, British Columbia, Canada) for a period of about three days to about 10 days, about 5 days to about 10 days, about 6 days to about 10 days, about 7 days to about 10 days, about eight days to about 10 days, or about nine days. In some embodiments, an alternative for StemSpan™ may be used. For example, a composition for the expansion of hemopoietic stem (HSPC) and/or progenitor cells of nonhuman primates may include IL3, IL6, FLT-3, TPO, and SCF (e.g., prior to transfection with reprogramming transcription factors). In some embodiments, animal serum (e.g., Fetal Bovine Serum) in the expansion medium may be replaced or combined with polyvinyl alcohol (PVA) to improve the efficiency of expansion of HSPC from nonhuman primates. CD34 is expressed not only by HSCs but by a multitude of other non-hematopoietic cell types, including muscle satellite cells, corneal keratocytes, interstitial cells, epithelial progenitors, and vascular endothelial progenitors. Thus, several different cell types may potentially exhibit progenitor activity and be expanded

Further, a method 700 for reprogramming PBMCs into iPSCs may include transfecting blood progenitor cells with transcription factors 720. The NHP PBMCs are reprogrammed into NHP induced pluripotent stem cells (iPSCs). The method for reprogramming the PBMCs into iPSCs may include transfecting the PBMCs with one or more transcription factors so that the cells are induced to overexpress the transcription factors. Transcription factors are one of the groups of proteins that read and interpret DNA. They bind to the DNA and help initiate a program of increased or decreased gene transcription. The stem cell derivation protocol is optimized for nonhuman primates specifically as there are different approaches taken to generate optimized stem cells for nonhuman primates. In some embodiments, transcription binding sites of these transcription factors may be mapped on individualized primate genomes to improve efficiency of the reprogramming process in NHPs, especially since transcription binding sites in human genomes are not completely conserved in NHP species. Thus, the use of human iPSCs reprogramming kits is not efficient for iPSC generation in NHP species. This is also important considering the fact that NHP genomes are very heterogeneous and existing mutations in NHP genomes result in inefficient binding of transcription factors (from human iPSC generation kits) in nonhuman primate cells.

Various transfection methods may be used, including but not limited to, electroporation, calcium-phosphate exposure, liposome-based transfection, viral-mediated transfection (also known as transduction), and the like. In an embodiment, a method for transfecting PBMCs includes transduction. Transduction may be accomplished using, for example, a Sendai virus, an adenoviral, an oncoretroviral, or a lentiviral vector. In some variations, transfection includes using a Sendai virus vector. In some instances, transfecting PBMCs includes liposome-mediated transfection. For example, the transcription factors may include, but not be limited to, c-myc, Klf4, Sox2, or Oct3/4. Additional or alternative transcription factors may be used and are within the scope of the present disclosure.

Further, a method 700 for reprogramming PBMCs into iPSCs may include co-culturing the cells (e.g., transfected and untransfected) with feeder cells 730. The feeder cells may comprise mouse embryonic fibroblast (MEF) cells, SNL feeder cells, or the like. The ratio of transfected cells to feeder cells may be about 1:2 to about 1:8, about 1:3 to about 1:7, about 1:4 to about 1:6, for example about 1:5. Co-culturing may occur on a subsequent day after transfection, for example about 20 hours to about 36 hours, about 18 hours to about 30 hours, about 18 hours to about 36 hours, about 22 hours to about 26 hours, etc. after transfection.

Co-culturing may occur in Essential-8™ medium (Thermofisher Scientific). In an embodiment, the Essential-8™ medium may include a tankyrase 1/2 inhibitor. The tankyrase 1/2 inhibitor may be present in the medium at a concentration of about 1 μM to about 3 μM, about 1.5 μM to about 2.5 μM, about 1 μM to about 2.5 μM, about 1.5 μM to about 3 μM, about 1.75 μM to about 2.25 μM, etc. In some embodiments, the tankyrase 1/2 inhibitor may maintain the NHP iPSCs in a pluripotent state and significantly reduce spontaneous differentiation of the NHP iPSCs.

Further, a method 700 for reprogramming PBMCs into iPSCs may include transferring unadhered cells to new medium. For example, the transfer may occur about 24 hours to about 72 hours, about 18 hours to about 78 hours, about 18 hours to about 30 hours, about 30 hours to about 42 hours, about 42 hours to about 52 hours, about 52 hours to about 66 hours, about 66 hours to about 78 hours, etc. after co-culturing. The unadhered cells may be cultured in a basal medium, for example, MEM medium, DMEM medium, RPMI medium, F12™ medium, and the like. Further, a method 700 for reprogramming PBMCs into iPSCs may optionally include adding medium to the adherent cells 740. The added media may comprise a medium that is feeder-free and xeno-free that supports the reprogramming of somatic cells and the spontaneous or directed differentiation of pluripotent stem cells (PSCs). For example, the added medium may be Essential-6@medium (Thermofisher Scientific). In an embodiment, adding medium occurs about 48 hours to 96 hours, about 42 hours to about 102 hours, etc. after transfection.

Further, a method 700 for reprogramming PBMCs into iPSCs may include optionally replacing the medium on the adherent cells. The medium may be replaced after about 90 hours to about 144 hours, about 96 hours to about 144 hours, about 90 hours to about 126 hours, about 114 hours to about 126 hours, etc. after transfection. The replacement medium may be a medium that is feeder-free and xeno-free that supports the reprogramming of somatic cells and the spontaneous or directed differentiation of pluripotent stem cells (PSCs). For example, the added medium may be Essential-6® medium (Thermofisher Scientific).

Further, a method 700 for reprogramming PBMCs into iPSCs may include optionally replacing the medium with medium comprising one or more growth factors 750. For example, the medium may comprise a medium that is feeder-free and xeno-free that supports the reprogramming of somatic cells and the spontaneous or directed differentiation of pluripotent stem cells (PSCs). For example, the added medium may be Essential-6@medium (Thermofisher Scientific). The growth factor may comprise basic fibroblast growth factor, DJ-1, epidermal growth factor, and the like. In an embodiment, the growth factor comprises basic fibroblast growth factor. The concentration of the growth factor (i.e., bFGF) may be about 100 ng/L to about 200 ng/L. In some embodiments, this particular growth factor and/or concentration is advantageous for the NHP iPSC derivation process since commercially available human bFGF is not as efficient in cross-reactivity with NHP cells. Medium replacement may occur about 144 hours to about 192 hours, about 160 hours to about 250 hours, about 162 hours to about 174 hours, about 188 hours to about 198 hours, about 210 hours to about 222 hours, about 232 hours to about 246 hours, etc. after transfection. Medium replacement may be repeated daily for one or more days or a plurality of days, for example, repeated once, twice, thrice, or more.

Further, a method 700 for reprogramming PBMCs into iPSCs may include optionally replacing the medium daily for one or more days or for a plurality of days 780. For example, the medium may be replaced between about 240 hours post-transfection to about 440 hours post-transfection. The medium may be replaced with basal medium, Essential-6®, Essential-8®, a medium comprising tumor growth factor-β (TGF-β) and/or b-FGF, or similar medium. In some embodiments, the medium may be replaced daily until visual identification of a first colony of iPSC.

Further, a method 700 for reprogramming PBMCs into iPSCs may include culturing the first colony of iPSC on a matrix 770, for example an extracellular matrix, Matrigel®, Cultrex®, Geltrex®, and the like. The first colony may be cultured in medium comprising a serine/threonine kinase inhibitor. For example, the medium may include Essential-8@. The serine/threonine kinase may include a Rho inhibitor or a Rho-associated coiled-coil containing protein kinase (ROCK) inhibitor, for example, Thiazovivin (TZV) or similar products. A concentration of the inhibitor may be about 1 μM to about 3 μM.

Further, a method 700 for reprogramming PBMCs into iPSCs may include passaging the cells after about 12 hours to about 48 hours. Passaging 760 may include refreshing the medium and adding a Wnt pathway inhibitor, tankyrase1/2 inhibitor, or the like. The inhibitor may be at a concentration of about 1 μM to about 3 μM, about 1.5 μM to about 2.5 μM, about 1 μM to about 2.5 μM, about 1.5 μM to about 3 μM, about 1.75 μM to about 2.25 μM, etc.

Optionally, iPSCs may be passaged and/or expanded by washing with a buffer (e.g., phosphate-buffered saline) and incubating the cells with a cell detachment solution. The cell detachment solution may comprise collagenase (or recombinant enzymes thereof), trypsin (or recombinant enzymes thereof), a chelating agent (e.g., ethylenediamine tetra-acetic acid), and the like.

Optionally, iPSCs may be expanded or maintained by culturing in medium on feeder cells or an extracellular matrix, as described elsewhere herein. The medium may comprise a serine/threonine kinase that may comprise a Rho inhibitor or a Rho-associated coiled-coil containing protein kinase (ROCK) inhibitor. The inhibitor may be at a concentration of about 1 nM to about 3 nM, about 1.5 μM to about 2.5 μM, about 1 μM to about 2.5 μM, about 1.5 μM to about 3 μM, about 1.75 μM to about 2.25 μM, etc.

In embodiments with subsequent passaging, the iPSCs may be incubated in a container, without feeder cells, the container having a substrate coated thereon. The substrate may be configured to retain pluripotency of the cells, a stable karyotype of the cells, and/or an expression of pluripotency markers by the cells. For example, the substrate may comprise laminin, a recombinant laminin fragment (e.g., 511 e8 fragment), vitronectin, recombinant vitronectin, or a combination thereof. The medium may comprise Essential-8®, E8-specific supplement (e.g., TGF-β, bFGF, etc.), and a Wnt pathway inhibitor, tankyrase 1/2 inhibitor, or the like. The inhibitor may be at a concentration of about 1 nM to about 3 nM, about 1.5 μM to about 2.5 μM, about 1 μM to about 2.5 μM, about 1.5 μM to about 3 μM, about 1.75 μM to about 2.25 μM, etc. The subsequent passaging may result in colonies with little-to-no differentiation.

Differentiation of NHP iPSCs into Neuronal Lineages

In some embodiments described herein, the methods may be used for differentiating NHP iPSCs to neuronal lineage cells, for example, NGN2 neurons, also described herein as induced neuronal cells or iN cells, or motor neurons. In some embodiments, the differentiation may be from NHP iPSCs to NGN2 neurons or iN cells. In some embodiments, the differentiation may be from NHP iPSCs to motor neurons. In some embodiments, the NHP species may include cynomolgus macaques (Macaca fascicularis), rhesus macaques (Macaca mulatta), and pig-tailed macaques (Macaca nemestrina).

In some embodiments, the differentiated neuronal lineage cells can be used for in vitro safety and efficacy assessments as well as discovery of human drugs in iPSC-derived neuronal lineage cells in NHPs.

To differentiate NHP iPSCs into neuronal lineage cells, for example, neural cells including NGN2 neurons (iN cells), or other neuronal lineage, a method may include optionally transitioning NHP iPSCs from a feeder-based culture condition (e.g., culturing iPSCs on MEF or other feeder cells) into a feeder-free condition. This transition into a feeder-free condition of NHP iPSCs may reduce or remove various small molecules that are secreted by the feeder cells into the culture media. For example, small molecules secreted by feeder cells can interrupt the process of differentiation of iPSCs to neural cells including NGN2 neurons.

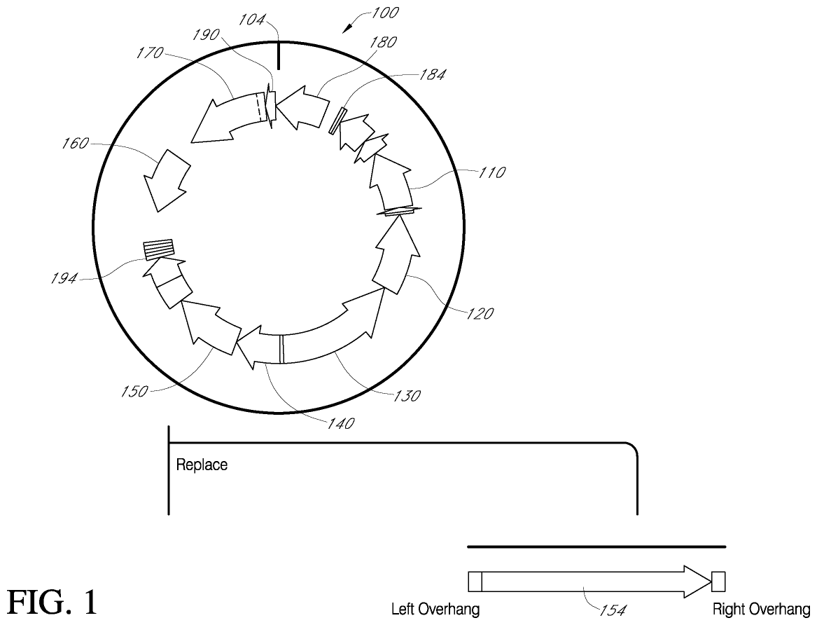

As shown in FIGS. 1-3 and FIG. 6, a vector 100, and associated method, may be used for differentiating NHP iPSCs into various cell types. The vector 100 (e.g., SEQ ID NO. 1) may be used for differentiating NHP iPSCs, for example, Macaca fascicularis iPSCs. The vector 100 may be a piggyBac vector. The vector 100 may include a nucleic acid sequence (e.g., SEQ ID NO. 7) encoding a selectable marker 110 (e.g., antibiotic resistance gene) for the selection of successfully transfected cells. For example, the nucleic acid sequence may encode the selectable marker 110 Puromycin (PuroR) between about 1113 base pairs and about 1712 base pairs as shown in SEQ ID NOs. 1. The vector 100 may include a nucleic acid sequence (e.g., SEQ ID NO. 14) encoding a modified rtTA protein 120 that binds to promoters containing the tet operator in the presence of doxycycline. For example, SEQ ID NO. 14 may be between about 1788 base pairs and about 2531 base pairs of SEQ ID NO. 1. The vector 100 may include a nucleic acid sequence (e.g., SEQ ID NO. 5) encoding a Tet-responsive promoter 140. For example, the Tet-responsive promoter 140 may be a unidirectional promoter (SEQ ID NO. 5), as shown in FIGS. 1-3, or a bi-directional promoter may be used, for example, to transcribe a reporter gene and a differentiation factor gene. The Tet-responsive promoter 140 may be between about 3815 base pairs and about 4193 base pairs as shown in SEQ ID NO. 1. The vector 100 may include a nucleic acid sequence (e.g., SEQ ID NOs. 8, 10) encoding one or more origins of replication (Ori) that enable the vector to be replicated. As shown in SEQ ID NO. 1, an f1 Ori 180 (e.g., SEQ ID NO. 10) may be between about 3 base pairs to about 458 base pairs for phage-based single-stranded DNA replication and packaging. Further, for example, as shown in SEQ ID NO. 1, an Ori 160 (e.g., SEQ ID NO. 8) may be between about 5852 base pairs to about 6440 base pairs for plasmid amplification in bacterial cells in preparation for transfection. The vector 100 may include a nucleic acid sequence (e.g., SEQ ID NO. 7) encoding a second selectable marker 170 (e.g., antibiotic resistance gene) for selection of those bacterial cells that are amplifying the vector. SEQ ID NO. 7 may be between about 6611 base pairs and about 7471 base pairs of SEQ ID NO. 1. For example, the nucleic acid sequence may encode Ampicillin (AmpR), which confers resistance to ampicillin, carbenicillin, and related antibiotics. The promoter 190 (e.g., SEQ ID NO. 11) for the selectable marker 170 may be between about 7472 base pairs and about 7576 base pairs of SEQ ID NO. 1. The vector 100 may optionally include a nucleic acid sequence (e.g., SEQ ID NOs. 12, 13) encoding a T7 promoter 184 for bacteriophage T7 RNA polymerase and/or a T3 promoter 194 for bacteriophage T3 RNA polymerase. For example, SEQ ID NO. 12 may be between about 623 base pairs and about 641 base pairs of SEQ ID NO. 1. SEQ ID NO. 13 may be between about 5411 base pairs and about 5429 base pairs of SEQ ID NO. 1. The vector 100 may include a nucleic acid sequence (e.g., SEQ ID NO. 6) for encoding a constitutive promoter 130 (e.g., Ubiquitin C promoter or UbC) to drive the expression of one or more differentiation factor genes in the target cells. For example, SEQ ID NO. 6 may be between about 2545 base pairs and about 3754 base pairs of SEQ ID NO. 1.

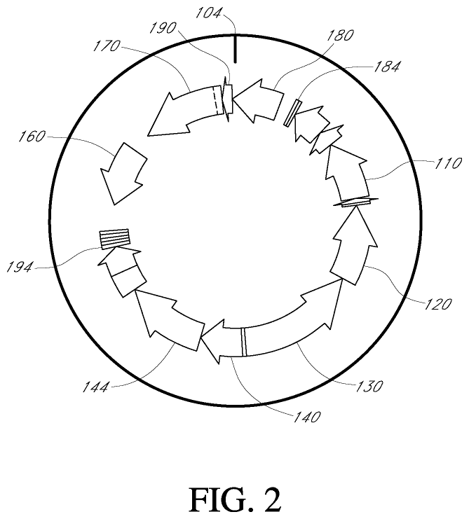

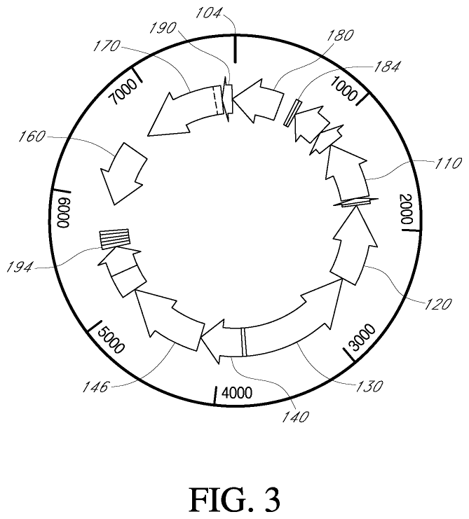

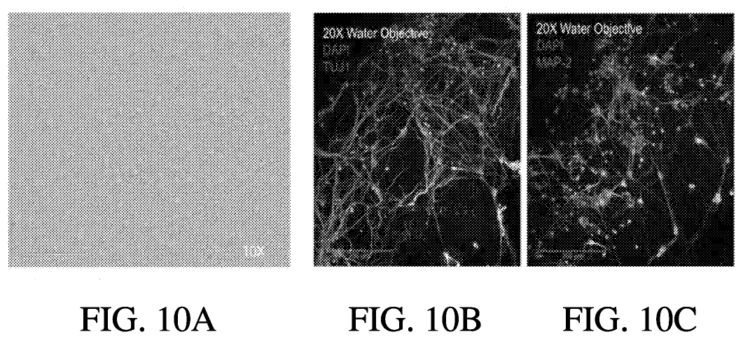

Producing a modified vector 100 may include replacing a reporter gene 150, for example, TurboGFP or SEQ ID NO. 4 (as shown in FIG. 1), with a differentiation factor gene 154 (as shown in FIGS. 2-3) or inserting a differentiation factor gene 154 upstream or downstream of a reporter gene 150 (as shown in FIGS. 6 and 10). For example, the reporter gene 150 may be used as a marker of transduction efficiency (i.e., cells that are not transduced may not survive in the presence of the compound used to transcribe the selectable marker). Although TurboGFP is shown and described herein, one of skill in the art will appreciate that other reporter genes may be used without departing from the intent and scope of the present disclosure. Alternative reporter genes include, but are not limited to, enhanced Green Fluorescent Protein (eGFP, excitation at 488 nm, emission at 507 nm), mCherry (excitation at 587 nm, emission at 610 nm), mVenus (excitation at 515 nm, emission at 528 nm), mKate2 (excitation at 588 nm, emission at 633 nm), TagRFP (excitation at 555 nm, emission at 584 nm), AmCyan1 (excitation at 458 nm, emission at 489 nm), mCerulean3 (excitation at 433 nm, emission at 475 nm), tdTomato (excitation at 554 nm, emission at 581 nm), mRuby2 (excitation at 559 nm, emission at 600 nm), sfGFP (Superfolder GFP, excitation at 485 nm, emission at 510 nm), and the like. The reporter gene 150 may be, for example, between about 4214 base pairs and about 4894 base pairs, as shown in SEQ ID NOs. 1 and 9.

The differentiation factor gene 154 may be Homo sapiens NGN2 144 or SEQ ID. 3 (as shown in FIG. 2) or Macaca fascicularis NEUROG2 146 or SEQ ID NO. 2 (as shown in FIG. 3), inserted into vector 100, for example a piggyBac vector backbone (e.g., SEQ ID NO. 1, OTPL007-pbvector). Although sequences for Macaca fascicularis are shown and described herein, one of skill in the art will appreciate that sequences derived from Homo sapiens may also apply and/or be used in the transduction of NHP iPSCs given the sequence similarity between NHP genomes and the Homo sapiens genome. To modify the vector 100 to express a differentiation factor gene, at least a portion of the reporter gene 150 may be replaced with SEQ ID NO. 2 directly, as shown in FIG. 3, or the reporter gene 150 may be replaced with SEQ ID. 3 directly, as shown in FIG. 2. In some embodiments, SEQ ID NO. 2 may be inserted into the vector 100 upstream or downstream of the reporter gene 150. In some embodiments, SEQ ID NO. 3 may be inserted into the vector 100 upstream or downstream of the reporter gene 150. Alternatively, to modify the vector 100 to express a differentiation factor gene, SEQ ID NO. 3 may be inserted in place of the reporter gene 150, as shown in FIGS. 2-3, and SEQ ID NO. 2 may replace SEQ ID NO. 3. SEQ ID NO. 2 may be positioned between about 4208 base pairs to about 5026 base pairs of vector 100. SEQ ID NO. 3 may be positioned between about 4208 base pairs and about 5033 base pairs in vector 100. In some embodiments, either SEQ ID NO. 2 or SEQ ID NO. 3 may be used to transfect NHP iPSCs due to the high sequence homology between SEQ ID NO. 2 and SEQ ID NO. 3.

The vector 100 may be modified using restriction enzymes, Gibson Assembly, or the like to replace the reporter gene 150 with the differentiation factor gene 154. For example, using Gibson Assembly, the differentiation factor gene and the vector 100 may each include overlapping sequences, complementary to each other. The vector 100 may be linearized (e.g., using restriction digestions or PCR amplification) to create open ends in the vector 100 for insertion of the gene. The gene and linearized vector may be mixed with an enzymatic reaction mixture, including for example, exonuclease, DNA polymerase, and DNA ligase. The reaction may be an isothermal reaction. For example, the reaction may be incubated at about 45 degrees to about 55 degrees to enable the gene to be inserted into the vector. The recombinant vector may be transformed into bacterial cells that can replicate the vector using the Ori in the vector. Bacterial cells with successful replication of the vector may be selected using a selectable marker, for example Ampicillin resistance (i.e., using the AmpR gene of the vector).

The vectors may respond to doxycycline induction while having continuous puromycin selection. The Tet-On 3G is a modified rTtA protein that binds tightly to the promoters containing the Tet operator in the presence of doxycycline. The vector 100 exhibits increased sensitivity to doxycycline, allowing for tighter control over gene expression levels compared to earlier Tet-On systems. By incorporating the puromycin resistance gene (PuroR), as shown in FIG. 2, alongside NGN2, cells expressing NGN2 (i.e., have been transduced) can be selectively cultured.

In some embodiments, a method 400 of transfecting NHP iPSCs may include transfecting the NHP iPSCs (e.g., electroporating the NHP iPSCs) with a vector expressing a differentiation factor gene 154 (e.g., any of SEQ ID NOs. 2-8, etc.) at block S410, and selecting for the transfected NHP iPSCs using a selectable marker (e.g., puromycin selection) at block S430, for example a selectable marker associated with vector 100. The method 400 may optionally further include expanding the transfected NHP iPSCs on a matrix at block S420, for example an extracellular matrix, Matrigel®, Cultrex®, Geltrex®, and the like. The transfected NHP iPSCs may be expanded in medium (e.g., E8 basal media+E8 Supplement containing thiazovivin). Method 400 may be used with vector 100, for example a piggyBac vector. Method 400 may also be used with a lentiviral vector, a Sendai virus vector, or an adenovirus vector, but with likely reduced transfection efficiency as compared to a piggyBac vector, as described elsewhere herein.

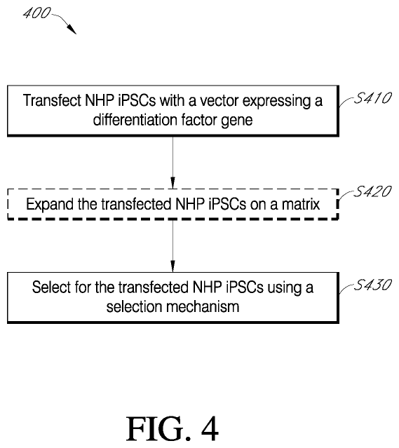

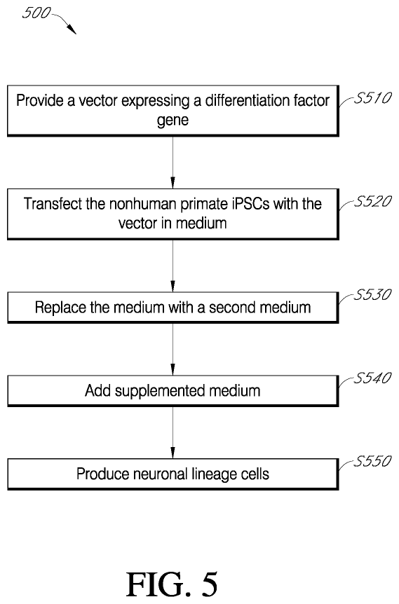

In some embodiments, as shown in FIG. 5, a method 500 of differentiating NHP iPSCs into neuronal lineage cells (e.g., iN cells) may include providing a vector expressing one or more differentiation factor genes (e.g., SEQ ID NOs. 2-3, etc.) at block S510; and transfecting the NHP iPSCs (e.g., electroporating the NHP iPSCs) with the vector at block S520. The vector 100 may be a modified vector, for example, a piggyBac vector backbone modified to express one or more differentiation factor genes. In some embodiments, the vector may be a lentiviral vector, a Sendai virus vector, or an adenovirus vector. As described above for FIG. 4, transfecting may further include selecting the transfected NHP iPSCs using a selectable marker.

As shown in FIG. 5, method 500 for differentiating NHP iPSCs into neuronal lineage cells (e.g., iN cells) may include replacing the medium with a second medium at block S530; adding supplemented medium at block S540; and producing neuronal lineage cells at block S550.

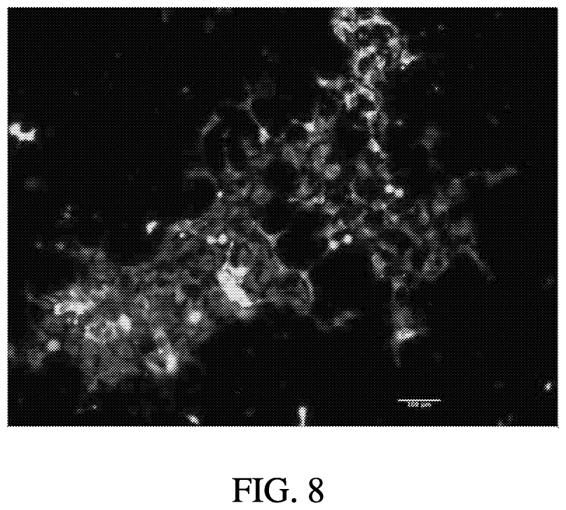

In some embodiments, replacing the medium with a second medium at block S530 may include replacing the culture medium with culture medium containing one or more of: brain-derived neurotrophic factor (BDNF), neurotrophin-3 (NT-3), and laminin. The BDNF, NT-3, and laminin may be human-derived. The BDNF, NT-3, and laminin may be mouse-derived. The second medium may include about 8 μg/L to about 12 μg/L, about 0.1 μg/L to about 20 μg/L, about 9 μg/L to about 11 μg/L, about 5 μg/L to about 15 μg/L, about 1 μg/L to about 15 μg/L, about 6 μg/L to about 14 μg/L, etc., BDNF. The second medium may include about 8 μg/L to about 12 μg/L, about 0.1 μg/L to about 20 μg/L, about 9 μg/L to about 11 μg/L, about 5 μg/L to about 15 μg/L, about 1 μg/L to about 15 μg/L, about 6 μg/L to about 14 μg/L, etc., NT-3. The second medium may include about 0.1 mg/L to about 0.4 mg/L, about 0.02 mg/L to about 2 mg/L, about 0.1 mg/L to about 0.3 mg/L, about 0.05 mg/L to about 0.5 mg/L, about 0.15 mg/L to about 0.25 mg/L, about 0.04 mg/L to about 1 mg/L, etc., laminin. The culture medium may include N2 supplement and nonessential amino acids (NEAA) in Dulbecco's Modified Eagle Medium F12 (DMEM/F-12), Roswell Park Memorial Institute (RPMI) 1640, Iscove's Modified Dulbecco's Medium (IMDM), Knockout® Serum Replacement (KOSR), or the like, as described elsewhere herein. Replacing the medium with a second medium may occur on about day negative one to about day one or on about day zero. Replacing the medium may include treating the NHP iPSCs with a cell detachment solution (e.g., enzyme-based); and plating the dissociated NHP iPSCs on about day one, about day zero, or about day negative one. In some embodiments, the dissociated NHP iPSCs are plated on matrix-coated plates (e.g., an extracellular matrix, Matrigel®, Cultrex®, Geltrex®, etc.) in medium (e.g., E8 basal media+E8 Supplement containing thiazovivin). In some embodiments, doxycycline may be added on about day negative one to about day one or on about day zero to induce TetO gene expression. In some embodiments, doxycycline is optionally retained in the medium until the end of the experiment. On or between about day negative one and about day two or on about day one, in some embodiments, a puromycin selection period (e.g., about 18 hours to about 30 hours) may be initiated. The puromycin selection period may be associated with block S430 of FIG. 4 or a separate selection period. On about day one to about day three, the method 500 at block S540 may include adding medium (e.g., Neurobasal) supplemented with L-glutamine or L-glutamine alternative containing BDNF and NT3. Optionally, and after about one day to about three days, about 20% to about 75% of the medium may be exchanged. This media exchange may be repeated every about two days. Neuronal lineage cells (e.g., iN cells) may be produced, as shown in block S550 of method 500 of FIG. 5, between about day 14 and about day 21. The efficiency of conversion of iPSCs into iN cells may be calculated, for example, by counts of cell densities in four random fields in a receptacle (e.g., well): (1) as the percentage of EGFP-positive transduced cells (e.g., as shown in FIG. 8) that also express MAP-2 (e.g., as shown in FIG. 10B) or neuronal nuclear antigen (NeuN); and/or (2) as the percentage of starting cells that become NeuN-positive.

FIG. 8 shows a fluorescence microscopy image of iPSCs from Macaca fascicularis transfected with a piggyBac vector expressing Macaca fascicularis NEUROG2. The transfected iPSCs are shown as expressing the reporter gene, green fluorescent protein (GFP). NHP iPSCs were expanded until they were about 90% confluent. NHP iPSCs were dissociated using an enzyme (e.g., accutase). The iPSCs were electroporated with an NGN2 expression vector (piggyBac vector) using the 4D-Nucleofector System (Lonza). After transfection, the NHP iPSCs were plated on extracellular matrix-coated plates and expanded. Puromycin selection was performed to isolate transfected cells. As shown in FIG. 8, transfection efficiency is 10% to about 30%, about 15% to about 20%, about 16% to about 18%, about 17% to about 23%, etc. Further viability of the cells after transfection with the expression vector was about 90% to about 100%.

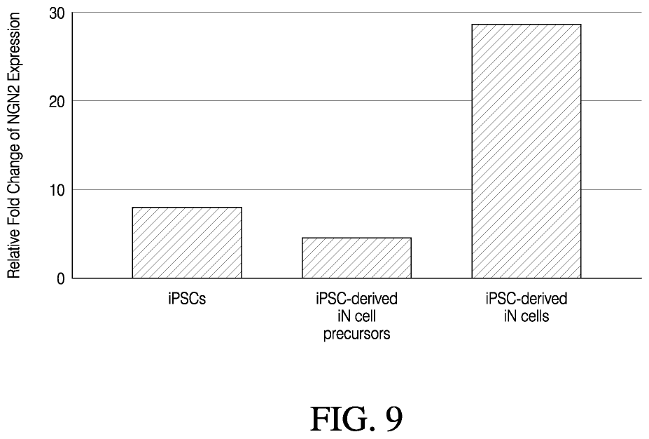

FIG. 9 shows quantitative polymerase chain reaction (qPCR). The iPSCs and NGN2-transfected, and NGN2 expression cells were cultured simultaneously. The transfected cells are doxycycline-inducible, and NGN2 is expressed upon addition of doxycycline in the culture media. All cells were collected, and RNA was extracted from the cells. Subsequently, cDNA was synthesized using a reverse transcription process, and NEUROG2 expression (fold change relative to glyceraldehyde-3-phosphate dehydrogenase, GAPDH, housekeeping gene) was quantified in various cell types of, or derived from, Macaca fascicularis. As shown in FIG. 9, Macaca fascicularis iPSCs (far left bar) express low levels of NEUROG2. Macaca fascicularis iPSC-derived iN cell precursors (middle bar) also express low levels of NEUROG2. In contrast, Macaca fascicularis iPSC-derived iN cells (far right bar) express abundant NEUROG2, indicating induction of NEUROG2 expression during neuronal differentiation of iPSC to iN cells.

FIG. 10A shows a brightfield image of iPSC-derived iN cells at 10× magnification. FIGS. 10B-10C show fluorescence microscopy images of iPSC-derived iN cells at 20× magnification. Cells were washed with 1×PBS, fixed with 4% formaldehyde in PBS for 15-20 minutes at room temperature, and washed 3 times with 1×PBS. The PBS was aspirated and 0.1% TritonX in DPBS was added to each well. The cells were incubated for 10 min in the TritonX, then washed 3× with DPBS for 5 minutes. The cells were blocked with a blocking buffer (10% Donkey Serum+0.1% Triton X in 1×PBS) for 1 hour at room temperature. The cells were stained with antibodies diluted in 1% Donkey Serum+0.1% Triton X in 1×PBS at 4 °C overnight. The cells were washed 3 times with washing buffer (0.1% Triton X in 1×PBS) and then stained with fluorophore-conjugated secondary antibodies diluted at 1:1000 (1% Donkey Serum+0.1% Triton X in 1×PBS) in the dark at room temperature for 1 hour. Finally, the cells were washed once with 100 ng/mL DAPI/PBS once and twice with 1×PBS before visualization. As shown in FIG. 10B, the cells were stained with DAPI as an indicator of cell viability and an antibody directed to TUJ1. TUJ1 is a marker of neurons in the central and peripheral nervous systems from the early stage of neural differentiation. The image in FIG. 10B shows expression of TUJ1 by the iPSC-derived iN cells, indicating neural differentiation. As shown in FIG. 10C, the cells were stained with DAPI as an indicator of cell viability and an antibody directed to MAP-2. MAP2 is a neuron-specific protein that stabilizes microtubules. The image in FIG. 10C shows expression of MAP-2 by the iPSC-derived iN cells, indicating neural differentiation.

FIG. 11 is a graph showing the number of cells successfully transfected, divided by the total number of cells used. As shown in FIG. 11, 0.17 means 170,000 cells out of 1 million cells were successfully transfected with the piggyBac vector, versus 0.006 means 6,000 cells out of 1 million cells were successfully transfected with the lentiviral vector.

### EXAMPLES

Embodiment 1. A vector for differentiating nonhuman primate iPSCs from Macaca fascicularis, wherein the vector comprises a nucleic acid sequence comprising a sequence encoding: a piggyBac vector backbone; and a differentiation factor gene.

Embodiment 2. The vector of embodiment 1, wherein the nonhuman primate iPSCs are differentiated into induced neuronal (iN) cells.

Embodiment 3. The vector of embodiment 1, wherein the differentiation factor gene is SEQ ID NO. 2.

Embodiment 4. The vector of embodiment 1, wherein the differentiation factor gene is SEQ ID NO. 3.

Embodiment 5. The vector of embodiment 1, wherein the piggyBac vector backbone further comprises a second sequence encoding a selectable marker and a third sequence encoding a promoter.

Embodiment 6. The vector of embodiment 1, wherein the differentiation factor gene is configured to be inserted into the piggyBac vector backbone using Gibson Assembly.

Embodiment 7. The vector of embodiment 1, wherein the differentiation factor gene is configured to be inserted into the piggyBac vector backbone using restriction enzymes.

Embodiment 8. The vector of embodiment 1, wherein the vector is configured to be electroporated into the nonhuman primate iPSCs.

Embodiment 9. The vector of embodiment 1, wherein differentiating comprises transfecting.

Embodiment 10. The vector of embodiment 1, wherein the piggyBac vector backbone comprises SEQ ID NO. 1.

Embodiment 11. The vector of embodiment 1, wherein the piggyBac vector backbone comprises a nucleic acid sequence encoding a selectable marker for selection of successfully transfected cells.

Embodiment 12. The vector of embodiment 11, wherein the selectable marker comprises a puromycin resistance marker.

Embodiment 13. The vector of embodiment 1, wherein the piggyBac vector backbone comprises a nucleic acid sequence encoding a modified rtTA protein that binds to promoters containing a tet operator in a presence of doxycycline.

Embodiment 14. The vector of embodiment 13, wherein the piggyBac vector backbone comprises a Tet-responsive promoter positioned between about 3815 base pairs and about 4193 base pairs of SEQ ID NO. 1.

Embodiment 15. The vector of embodiment 1, wherein the piggyBac vector backbone comprises a constitutive promoter configured to drive expression of one or more differentiation factor genes in target cells.

Embodiment 16. The vector of embodiment 15, wherein the constitutive promoter comprises SEQ ID NO. 6 positioned between about 2545 base pairs and about 3754 base pairs of SEQ ID NO. 1.

Embodiment 17. The vector of embodiment 1, wherein the differentiation factor gene is configured to replace at least a portion of a reporter gene in the piggyBac vector backbone.

Embodiment 18. The vector of embodiment 1, wherein the differentiation factor gene is configured to be inserted upstream or downstream of a reporter gene in the piggyBac vector backbone.

Embodiment 19. The vector of embodiment 1, wherein the piggyBac vector backbone comprises SEQ ID NO. 1 and a constitutive promoter comprising SEQ ID NO. 6 positioned between about 2545 base pairs and about 3754 base pairs of SEQ ID NO. 1 to drive expression of one or more differentiation factor genes in target cells.

Embodiment 20. The vector of embodiment 1, wherein the differentiation factor gene comprises Macaca fascicularis NEUROG2, wherein the Macaca fascicularis NEUROG2 is configured to replace at least a portion of a reporter gene in the piggyBac vector backbone and is positionable between about 4208 base pairs and about 5026 base pairs of the piggyBac vector backbone.

References in the specification to “one embodiment,” “an embodiment,” “an illustrative embodiment,” “some embodiments,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

As used in the description and claims, the singular form “a”, “an” and “the” include both singular and plural references unless the context clearly dictates otherwise. For example, the term “cell” may include, and is contemplated to include, a plurality of cells. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A vector for differentiating nonhuman primate iPSCs from Macaca fascicularis, wherein the vector comprises a nucleic acid sequence comprising a sequence encoding:
a piggyBac vector backbone; and
a differentiation factor gene.

2. The vector of claim 1, wherein the nonhuman primate iPSCs are differentiated into induced neuronal (iN) cells.

3. The vector of claim, wherein the differentiation factor gene is SEQ ID NO. 2.

4. The vector of claim, wherein the differentiation factor gene is SEQ ID NO. 3.

5. The vector of claim, wherein the piggyBac vector backbone further comprises a second sequence encoding a selectable marker and a third sequence encoding a promoter.

6. The vector of claim, wherein the differentiation factor gene is configured to be inserted into the piggyBac vector backbone using Gibson Assembly.

7. The vector of claim, wherein the differentiation factor gene is configured to be inserted into the piggyBac vector backbone using restriction enzymes.

8. The vector of claim, wherein the vector is configured to be electroporated into the nonhuman primate iPSCs.

9. The vector of claim, wherein differentiating comprises transfecting.

10. The vector of claim, wherein the piggyBac vector backbone comprises SEQ ID NO. 1.

11. The vector of claim 1, wherein the piggyBac vector backbone comprises a nucleic acid sequence encoding a selectable marker for selection of successfully transfected cells.

12. The vector of claim 11, wherein the selectable marker comprises a puromycin resistance marker.

13. The vector of claim 1, wherein the piggyBac vector backbone comprises a nucleic acid sequence encoding a modified rtTA protein that binds to promoters containing a tet operator in a presence of doxycycline.

14. The vector of claim 13, wherein the piggyBac vector backbone comprises a Tet-responsive promoter positioned between about 3815 base pairs and about 4193 base pairs of SEQ ID NO. 1.

15. The vector of claim 1, wherein the piggyBac vector backbone comprises a constitutive promoter configured to drive expression of one or more differentiation factor genes in target cells.

16. The vector of claim 15, wherein the constitutive promoter comprises SEQ ID NO. 6 positioned between about 2545 base pairs and about 3754 base pairs of SEQ ID NO. 1.

17. The vector of claim 1, wherein the differentiation factor gene is configured to replace at least a portion of a reporter gene in the piggyBac vector backbone.

18. The vector of claim 1, wherein the differentiation factor gene is configured to be inserted upstream or downstream of a reporter gene in the piggyBac vector backbone.

19. The vector of claim 1, wherein the piggyBac vector backbone comprises SEQ ID NO. 1 and a constitutive promoter comprising SEQ ID NO. 6 positioned between about 2545 base pairs and about 3754 base pairs of SEQ ID NO. 1 to drive expression of one or more differentiation factor genes in target cells.

20. The vector of claim 1, wherein the differentiation factor gene comprises Macaca fascicularis NEUROG2, wherein the Macaca fascicularis NEUROG2 is configured to replace at least a portion of a reporter gene in the piggyBac vector backbone and is positionable between about 4208 base pairs and about 5026 base pairs of the piggyBac vector backbone.
